(19) United States
(12) Patent Application Publication
(10) Pub. No.: US 2009/0018088 A1
(43) Pub. Date: Jan. 15, 2009

(54) TREATING CANCER WITH CARDIAC GLYCOSIDES

(75) Inventors: Roland Valdes, JR., Simpsonville, KY (US); Kenneth Ihenetu, Louisville, KY (US); Rafael Fernandes-Botran, Louisville, KY (US); Hassan Qazzaz, Louisville, KY (US)

Correspondence Address: VIKSNINS HARRIS & PADYS PLLP, P.O. BOX 111098, ST. PAUL, MN, 55111-1098, US

(73) Assignee: University of Louisville Research Foundation, Louisville, KY (US)

(21) Appl. No.: 12/131,763

(22) Filed: Jun. 2, 2008

Related U.S. Application Data
(63) Continuation of application No. PCT/US2006/042014, filed on Oct. 27, 2006.

(51) Int. Cl. A61K 31/704; A61K 31/70; C12N 5/00; A61P 35/00

(52) U.S. Cl. 514/26; 514/23; 435/375

(57) ABSTRACT

The invention provides methods to treat cancer with cardiac glycosides.

### RELATED APPLICATIONS

This application claims the benefit of priority of U.S. Provisional Application No. 60/799,199, filed May 9, 2006 and of PCT/US2006/042014, filed on Oct. 27, 2006. The entire content of these applications is hereby incorporated herein by reference.

### STATEMENT OF GOVERNMENT SUPPORT

Work related to this application was funded by the U.S. government (NIH Grant HL-59404). The government has certain rights in this application.

### BACKGROUND

Most treatment plans for patients with cancer include surgery, radiation therapy, and/or chemotherapy. However, because of problems with such treatment plans, such as side-effects caused by radiation therapy and chemotherapy, additional methods are needed for treating cancer.

### SUMMARY OF CERTAIN EMBODIMENTS OF THE INVENTION

It has been discovered that cardiac glycosides, such as digoxin and ouabain, induce apoptosis and have anticancer properties. Accordingly, certain embodiments of the present invention provide methods for treating cancer in a subject, comprising administering to the subject an effective amount of a cardiac glycoside so as to treat the cancer.

Certain embodiments of the present invention provide methods for inducing cellular apoptosis of a cancerous cell, comprising contacting the cancerous cell with an effective apoptosis-inducing amount of a cardiac glycoside.

Certain embodiments of the present invention provide methods for increasing the anticancer effects of a cancer therapy on a cancerous cell, comprising contacting the cancerous cell with an effective amount of a cardiac glycoside prior to administering the cancer therapy.

### BRIEF DESCRIPTION OF THE FIGURES

FIG. 1. Flow cytometric analysis showing induction of apoptosis. Upper two panels show Jurkat cells not exposed and exposed to ultraviolet radiation for 48 hours. Lower 3 panels show cells responding to exposure to treatment with increasing concentrations of digoxin. Note the increase in cell density in the upper right hand quadrant, which is characteristic of early apoptosis.

FIG. 2. Induction of apoptosis in Jurkat cells treated with digoxin and ouabain. Jurkat cells were exposed to ultraviolet radiation digoxin or ouabain for 48 h at the indicated concentrations. Percent apoptosis was determined by flow cytometry as in FIG. 1 (percent of cells in early and late apoptosis relative to controls). The means and SEM of four separate experiments are shown. Asterisks denote significant difference (P<0.05) from untreated control (student's t-test).

FIG. 3. Induction of apoptosis in Daudi cells treated with digoxin and ouabain. Daudi cells were exposed to ultraviolet radiation, digoxin or ouabain for 48 h at the indicated concentrations. Percent apoptosis was determined by flow cytometry as in FIG. 1 (percent of cells in early and late apoptosis relative to controls). The means and SEM of four separate experiments are shown. Asterisks denote significant difference (P<0.05) from untreated control (student's t-test).

FIG. 4. Resistance of K 562 cells to induction of apoptosis after treatment with digoxin and ouabain. K 562 cells were treated and analyzed as in prior Figures. Results are of four separate experiments.

FIG. 5. Resistance of peripheral blood mononuclear cells (PBMC) to induction of apoptosis after treatment with digoxin and ouabain. PBMC were treated and analyzed as in prior Figures. Results of four separate experiments.

FIG. 6. Effect of cardiac glycoside on DEVD-dependent Caspase-3 activity. Tumor cell lines (1×10⁷ cells/mL) and PBMC (1×10⁷ cells/mL) were exposed to ultraviolet irradiation or digoxin (100 nM) for 12 h. Results represent measurement of Caspase-3 activity relative to the untreated controls. Three independent experiments differed by less than 10%.

FIG. 7. Selective pro-apoptotic effect of digoxin on Jurkat cells compared to PBMCs when challenged with phytohemagglutinin (PHA). Jurkat cells (5×10⁵/mL) were stimulated with PHA (1 μg/mL) before exposure to digoxin (10 nM and 100 nM), for 48 h. Digoxin synergistically increased the percent of apoptosis in Jurkat cells in the presence of PHA. No such effect was observed in the case of PBMC. Results of four independent experiments (mean and SEM) are shown. Asterisk denotes statistically significant difference relative to control (P<0.05).

### DETAILED DESCRIPTION

It has been discovered that cardiac glycosides (e.g., digoxin and ouabain) at non-toxic concentrations induce apoptosis in human lymphoblastic cell lines in vitro. At the concentrations tested, these drugs did not induce apoptosis in a human pro-erythroblastoid leukemic cell line or in isolated normal peripheral blood mononuclear cells in vitro. The human cell lines studied included: human T-cell lymphoblastic cell line (Jurkat E6-1); human B-cell Burkitt's lymphoma cell line (Daudi); human pro-erythroblastoid leukemic cell line (K 562). Apoptosis was estimated by flow cytometric analysis following Annexin V-FITC and propidium iodide staining (Vermes et al., J Immunol. Methods, 184, 39-51 (1995)), and confirmed by activation of DEVD-dependent caspase 3 activities (Gurtu et al., Analytical Biochemistry, 251, 98-102 (1997)). Cardiac glycosides were not only effective in inducing apoptosis in human leukemic and lymphoblastic cell lines but were also specific and sensitive at non-toxic concentrations. Taken together, this data suggest for the first time that cardiac glycosides can be used as specific and sensitive agents to target cancers, for example, of lymphoblastic origin.

Accordingly, certain embodiments of the present invention provide methods for treating cancer in a subject (e.g., a mammal such as a human), comprising administering to the subject an effective amount of a cardiac glycoside so as to treat the cancer.

Certain embodiments of the present invention provide methods for inducing cellular apoptosis of a cancerous cell, comprising contacting the cancerous cell with an effective apoptosis-inducing amount of a cardiac glycoside.

Certain embodiments of the present invention provide methods for increasing the anticancer effects of a cancer therapy on a cancerous cell, comprising contacting the cancerous cell with an effective amount of a cardiac glycoside prior to administering the cancer therapy. For example, the effectiveness of the cancer therapy may be increased to a level above the effectiveness demonstrated without the cardiac glycoside. In some embodiments, the effect(s) of the cardiac glycoside will enable the dosage of the cancer therapy to be decreased and to thereby decrease the side-effects of the cancer therapy.

In some embodiments of the invention, the contacting step occurs in vivo.

In some embodiments of the invention, the contacting step occurs in vitro.

In some embodiments of the invention, the cardiac glycoside is a cardenolide. In some embodiments of the invention, the cardenolide is a digoxigenin, digoxin, dihydrodigoxin, digitoxigenin, digitoxin, neriifolin, strophanthidin, convallatoxin, acetylstrophanthidin, ouabagenin, or ouabain.

In some embodiments of the invention, the cardenolide is a mammalian cardenolide.

In some embodiments of the invention, the cardiac glycoside is a digoxin-like factor (DLF), digoxin-like immunoreactive factor (DLIF), ouabain-like factor (OLF), dihydroouabain-like factor (Dh-OLF), or dihydrodigoxin-like factor (Dh-DLIF).

In some embodiments of the invention, the cardiac glycoside is a bufadienolide. In some embodiments of the invention, the bufadienolide is a bufalin, proscillardin, marinobufagenin, cinobufagen, or cinobufatolin.

In some embodiments of the invention, the method further comprises administering an additional cancer therapy to the subject.

In some embodiments of the invention, the method further comprises administering an additional cancer therapy to the cell.

In some embodiments of the invention, the additional cancer therapy is chemotherapy or radiation.

In some embodiments of the invention, the effective amount of the cardiac glycoside (e.g., that is administered to the subject or contacted with the cell) does not significantly inhibit the activity of the sodium pump. For example, the effective amount causes an inhibition of less than 100% (e.g., less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5%) of the activity of the sodium pump.

In some embodiments of the invention, the cancer is breast cancer, prostate cancer, lung cancer, colon cancer, hepatic cancer, skin cancer, leukemia, or lymphoma.

Certain embodiments of the present invention provide pharmaceutical compositions comprising an effective anticancer amount of a cardiac glycoside and a pharmaceutically acceptable carrier.

Certain embodiments of the present invention provide uses of a cardiac glycoside to prepare a medicament useful for treating cancer in an animal.

Certain embodiments of the present invention provide uses of a cardiac glycoside to prepare a medicament useful for inducing cellular apoptosis of a cancerous cell.

Certain embodiments of the present invention provide uses of a cardiac glycoside to prepare a medicament useful for increasing the anticancer effects of a cancer therapy on a cancerous cell.

Certain embodiments of the present invention provide pharmaceutical compositions that comprise a cardiac glycoside that are useful for treating cancer, inducing cellular apoptosis of a cancerous cell, and/or increasing the anticancer effects of a cancer therapy on a cancerous cell. Such a composition may comprise an amount of the cardiac glycoside that is effective for the intended purpose but that does not significantly inhibit the activity of the sodium pump.

In some embodiments of the invention, the production of an endogenous compound (e.g., a cardenolide) is regulated so as to control the endogenous production (i.e., administration) of the compound.

Cardiac glycosides generally include three structures: a steroid nucleus and an unsaturated lactone (together referred to as aglycone) and a carbohydrate. A cardiac glycoside may be, e.g., a cardenolide or a bufadienolide. Cardenolides have a five-membered lactone ring (e.g., an unsaturated butyrolactone ring) attached to the steroid, whereas the bufadienolides have a six-membered lactone ring (e.g., an a-pyrone ring) attached to the steroid. As used herein, a cardiac glycoside may be, e.g., a mammalian cardiac glycoside or a plant cardiac glycoside. Mammalian cardiac glycosides have structures similar to plant cardiac glycosides, but may be endogenously produced in mammals. In certain embodiments, the cardiac glycoside is oxidized or reduced. In certain embodiments, the cardiac glycoside is deglycosylated. (See, e.g., Qazzaz et al., Arch Biochem Biophys, 328(1), 193-200 (1996); Qazzaz et al., J Biol Chem, 271(15) 8731-8737 (1996); Qazzaz et al., Clin Chem, 42(7), 1092-1099 (1996); Qazzaz et al., Biochim Biophys Acta, 1472(3), 486-497 (1999); Qazzaz et al., Endocrinology, 141(9), 3200-3209 (2000); El-Masri et al., Clin Chem, 48(10), 1720-1730 (2002); Qazzaz et al, Clin Chem, 50(3), 612-620 (2004); Jortani et al, Crit Rev Clin Lab Sci, 34(3), 225-274 (1997); Jortani et al., Cardiovasc Toxicol, 1(2), 165-170 (2001); Pullen et al., J Pharmacol Exp Ther, 310(1), 319-325 (2004); Schoner, Eur J Biochem, 269(10), 2440-2448 (2002); and U.S. Pat. No. 6,835,715.) The art worker may obtain cardiac glycosides, e.g., from their natural source or they may be synthesized.

The terms “treat” and “treatment” refer to both therapeutic treatment and prophylactic or preventative measures, wherein the object is to prevent or decrease an undesired physiological change or disorder, such as the development or spread of cancer. For purposes of this invention, beneficial or desired clinical results include, but are not limited to, alleviation of symptoms, diminishment of extent of disease, stabilized (i e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The cardiac glycoside may be administered by any route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal.

The dosage of the cardiac glycoside(s) will vary depending on age, weight, and condition of the subject. Treatment may be initiated with small dosages containing less than optimal doses, and increased until a desired, or even an optimal effect under the circumstances, is reached. In general, the dosage is about 1 μg/kg up to about 100 μg/kg body weight, e.g., about 2 μg/kg to about μg/kg body weight of the subject, e.g., about 8 μg/kg to about 35 μg/kg body weight of the subject. Higher or lower doses, however, are also contemplated and are, therefore, within the confines of this invention. A medical practitioner may prescribe a small dose and observe the effect on the subject's symptoms. Thereafter, he/she may increase the dose if suitable. In general, the cardiac glycoside is administered at a concentration that will afford effective results without causing any unduly harmful or deleterious side effects, and may be administered either as a single unit dose, or if desired in convenient subunits administered at suitable times.

A pharmaceutical composition of the invention is formulated to be compatible with its intended route of administration. For example, the therapeutic agent may be introduced directly into the cancer of interest via direct injection. Additionally, examples of routes of administration include parenteral, e.g., intravenous, intradermal, subcutaneous, oral (e.g., ingestion or inhalation), transdermal (topical), transmucosal, and rectal administration. Such compositions typically comprise the cardiac glycoside and a pharmaceutically acceptable carrier. As used herein, “pharmaceutically acceptable carrier” is intended to include any and all solvents, dispersion media, coatings, antibacterial and anti-fungal agents, isotonic and absorption delaying agents, and the like, compatible with pharmaceutical administration. The use of such media and agents for pharmaceutically active substances is well known in the art.

Solutions or suspensions can include the following components: a sterile diluent such as water for injection, saline solution (e.g., phosphate buffered saline (PBS)), fixed oils, a polyol (for example, glycerol, propylene glycol, and liquid polyetheylene glycol, and the like), glycerine, or other synthetic solvents; antibacterial and antifungal agents such as parabens, chlorobutanol, phenol, ascorbic acid, thimerosal, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol or sorbitol, and sodium chloride in the composition. Prolonged administration of the injectable compositions can be brought about by including an agent that delays absorption. Such agents include, for example, aluminum monostearate and gelatin. The parenteral preparation can be enclosed in ampules, disposable syringes, or multiple dose vials made of glass or plastic.

It may be advantageous to formulate compositions in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms of the invention are dependent upon the amount of a compound necessary to produce the desired effect(s). The amount of a compound necessary can be formulated in a single dose, or can be formulated in multiple dosage units. Treatment may require a one-time dose, or may require repeated doses.

The effects of cardiac glycosides on the induction of apoptosis in two human leukemic lymphoblastic cell lines, on human erythroleukoblastoid cells, and on normal human peripheral blood mononuclear cells (PBMC) was investigated. The effect of phytohemagglutinin (PHA) to promote activation-induced apoptosis in the presence or absence of cardiac glycosides was also investigated. Apoptosis was measured by flow cytometric analysis after staining the cells with annexin V/propidium iodide and confirmed by caspase-3 activity assay. Statistical evaluation was performed using Student's t-test. Differences were considered significant at P≦0.05.

It was discovered that exposure of cancer cells to cardiac glycosides such as digoxin and ouabain led to a reduction in cell viability and a concentration-dependent induction of apoptosis when compared with untreated controls. Within the concentration range of cardiac glycoside tested (10 nM-500 nM), the highest degree of apoptosis, as a percentage of cells displaying apoptotic characteristics by flow cytometry, were: Jurkat cells {digoxin (500 nM=50.2±4.5%); ouabain (100 nM=47.6±3.9%)} and Daudi cells {digoxin (500 nM=83.2±7.3%); ouabain (500 nM=81.1±6.1%)}. In contrast, neither digoxin nor ouabain significantly induced apoptosis in K 562 cells or in PBMCs when compared with untreated controls at comparable cardiac glycoside concentrations. Further, the presence of cardiac glycosides selectively increased the sensitivity of Jurkat cells to PHA-induced apoptosis by 50% when compared to PBMCs treated in a similar manner. Surprisingly, the concentrations of the cardiac glycoside (e.g., digoxin and ouabain (20 to 40 nM)) needed to induce a 50% apoptotic response, based on the maximum amount of apoptosis achieved at a cardiac glycoside concentration of (500 nM) for each of the cancer cell lines, was considerably lower than the IC₅₀ needed to inhibit sodium ATPase activity in porcine cerebral cortex (PCC): digoxin {(IC₅₀=910 nM, range 820-1010 nM, n=3); and ouabain (IC₅₀=600 nM, range 550-650 nM, n=5)}.

Thus, low nanomolar concentrations of cardiac glycosides such as digoxin and ouabain induce apoptosis in human T cell lymphoblastic and B cell lymphoblastic (Burkitt's lymphoma) cancer cells but not in normal human peripheral blood leukocytes or erythroblastoid leukemia cells. The cardiac glycosides synergistically increased the ability of PHA to induce apoptosis in Jurkat cells but not in PBMC. These results indicate that these cancer cells are selective and sensitive to induction of apoptosis either through partial inhibition of Na,K-ATPase or by an alternative mechanism other than direct inhibition of sodium pump activity. Thus, cardiac glycosides are cell-selective anticancer compounds.

Plant-derived cardiac glycosides such as digoxin are clinically indicated for their anti-dysrhythmic effects. Their main pharmacological actions are mediated through interaction with the sodium pump, Na⁺,K⁺-ATPase (NKA). Inhibition of the sodium pump by cardiac glycosides at therapeutic concentrations produces a positive ionotropic effect mediated by rises in intracellular calcium [Ca]ᵢ²⁺ with a resultant increase in cardiac contractility. Recently, endogenous mammalian cardiotonic steroids known as digoxin-like immunoreactive factors (DLIF) and ouabain-like factors (OLF), which are secreted by the adrenal glands and are believed to constitute a hormonal-axis regulating the activity of the sodium pump, have been identified (Qazzaz et al., Clin Chem, 50, 469-470 (2004)).

The Na⁺,K⁺-ATPase is centrally important as a transport-protein for maintaining the high intracellular K⁺ and low intracellular Na⁺ in the cytoplasm required for normal membrane potential. This ionic equilibrium is important for cell growth, differentiation and cell survival. Apoptosis or programmed cell death is responsible for homeostatic removal of cells and is implicated in mediating pathological cell loss in many disease states ranging from cancer to inflammation. To date, it has not been clear whether inhibition of the Na⁺,K⁺-ATPase could induce apoptosis in normal or transformed cells, particularly those from the immune system.

The possibility of selective induction of apoptosis was investigated using two human lymphoma cell lines: an acute T-cell lymphoblastic leukemic cell line (Jurkat E6-1) and a B cell Burkitt's lymphoma cell line (Daudi). The effects of cardiac glycosides in these cells were compared to their action on an erythroblastoid leukemic cell line (K562) and normal human peripheral blood mononuclear cells (PBMC). The results demonstrated that cardiac glycosides (e.g., digoxin and ouabain) induced apoptosis in human lymphoma cell lines in a concentration dependent manner but not in erythroblastoid leukemic cells or normal human peripheral blood mononuclear cells. These drugs also selectively synergized with a mitogenic stimulus (e.g., phytohemagglutinin (PHA)) to induce apoptosis in cancer cells (e.g., Jurkat cells). Surprisingly, the induction of apoptosis by cardiac glycosides occurs at concentrations much lower than those typically required to inhibit Na⁺,K⁺-ATPase in vitro.

The pharmacological actions of cardiac glycosides have been extensively studied (for a more recent review, refer to Schoner et al., Sem. Nephrol, 25, 343-351 (2005) and Wasserstrom et al., Am J Physiol.-Heart Circ Physiol, 289, H1781-H1793 (2005)). The Na⁺,K⁺-ATPases are the well known specific targets for the cardiac glycosides (e.g., digitalis) and their related congeners (Skou et al., J Bioenerg Biomembr, 24, 249-261 (1992)). The net effect of their binding to NKA at therapeutic concentrations is an extensive increase in cardiac contractility, mainly in the diseased heart. This effect is exploited pharmacologically in the treatment of cardiac arrythmias. These positive ionotropic effects are explained by an increase in the intracellular calcium concentrations in myocardial cells. Whether the same biochemical mechanisms underlying the effects of cardiac glycosides in myocardial cells also play a role in non-excitable cells such as those from immune hematological origin is not known.

In the current study, the sensitivity of cardiac glycosides on induction of apoptosis in lymphoblastic cancer cells was examined. The efficacy of digoxin and ouabain in inducing apoptosis in these cell lines was compared with NKA catalytic inhibition activity using porcine cerebral cortex (Table 1). The porcine cerebral cortex (PCC) is a model system known to express the three isoforms of NKA consisting of the three different alpha subunits (α1, α2 and α3) (Rose et al., Clin Chem, 40, 1674-1685 (1994)). The alpha subunit of the NKA contains the binding site of the cardiac glycosides, and like most cationic transporter proteins, the beta subunit act as chaperon, stabilizing the correct assembly of the alpha subunit and facilitating the delivery of the protein to the plasma membrane (Blanco et al., Am J Physiol, 275 (Renal Physiol. 44), F633-F650 (1998)). Surprisingly, the potency of cardiac glycosides for inducing apoptosis in lymphoblastic cancer cells was at least 20-fold higher than that needed to inhibit the NKA catalytic activity in the porcine cerebral cortex (Table 1).

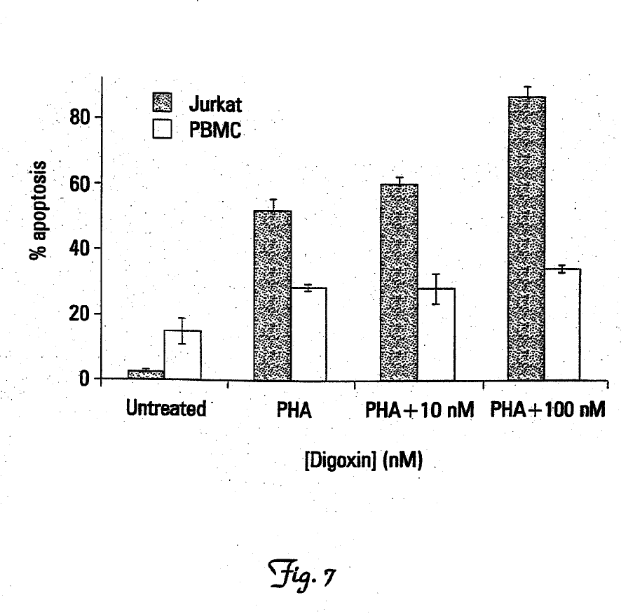

While the cardiac glycosides employed in our studies induced apoptosis in malignant lymphoblastic cell lines, no such effects were seen in the pro-erythroblastoid cell line or normal human peripheral blood mononuclear cells. Of particular note was the fact that the resistant cells (pro-erythroblastoid cells and PBMC) were also resistant to induction of apoptosis by ultraviolet irradiation. This finding raised the question of specificity of cardiac glycosides on the susceptible lymphoblastic cells to treatment with cardiac glycosides. In order to address this issue, the effects of digoxin on induction of apoptosis in the acute T cell lymphoblastic leukemic cell line (Jurkat) and PBMC following stimulation with PHA were compared. It is of note that the PBMC employed in this study consisted of at least 95% lymphocytes, suggesting that the effects observed in these cells are mostly effects on normal peripheral blood lymphocytes. It has been shown that activation induced cell death (AICD) in mature normal T lymphocytes and T cell leukemic cell line is mediated by Fas/FasL interaction (Martinez-Lorenzo et al., Immunology, 89, 511-517 (1996); Ju et al., Nature, 373, 444-448 (1995); and Alderson et al., J Exp Med, 181, 71-77 (1995)). Resting T lymphocytes constitutively express Fas but not FasL. Upon stimulation with PHA, a T cell receptor ligand, FasL expression is induced and Fas/FasL interaction leads to apoptosis (Liu et al., Biochem Biophys Res Com, 260, 562-567 (1999)). Interestingly, digoxin synergistically induced apoptosis in the acute T-lymphoblastic leukemic cell line (FIG. 7). No such synergistic effect was seen in the normal peripheral blood mononuclear cells, indicating that the effect observed with digoxin was indeed specific for the T-leukemic cells.

It was recently recognized that endogenous factors similar in structure to the plant-cardiac glycosides exist in mammals. These endogenous compounds, referred to as DLIF and OLF, are synthesized by the adrenal glands (Qazzaz et al., Clin Chem, 50, 469-470 (2004)). The physiologic function of these mammalian cardiac glycosides remains unclear, particularly because the reported concentration of these mammalian-derived cardenolides in blood appear to be 10 to 100 times lower (Qazzaz et al., Clin Chem, 42, 1092-1099 (1996); Qazzaz et al., J Biol. Chem, 271, 8731-8737 (1996b); and El-Masri et al., Clin Chem; 48-10, 1720-1730 (2002)) than the therapeutic concentrations of digoxin in blood, which is approximately 2 nM (Kometiani et al., Mol Pharmacol, 67, 929-936 (2005)). Thus, an endogenous mechanism may exist that regulates apoptosis through selective destruction of transformed cells as they are produced in vivo. This hypothesis suggests that subjects with appropriate amounts of DLIF or OLF in their blood would be protected from development and proliferation of spontaneously transformed cells by making them more susceptible to elimination by apoptosis than would be the normal cells surrounding them. Thus, cardiac glycosides may play a protective role and may be useful as natural adjuncts to cancer therapy by selectively sensitizing cancer cells to destruction, e.g., to natural physiologic destruction or destruction by administered chemotherapeutic agents. Pretreating or concurrently dosing subjects with cardiac glycosides, e.g., at low yet effective concentrations below those that would risk toxic cardiac responses or that would affect the sodium pump, would also be useful.

In summary, the results presented herein point to several important findings: a) plant-derived cardiac glycosides, such as digoxin and ouabain at non-toxic concentrations, induce apoptosis in transformed cells and not in normal cells; b) the presence of these glycosides increases the sensitivity of transformed cells to the pro-apoptotic effects of a mitogenic challenge such as PHA; and, c) the concentrations of cardiac glycosides at which the pro-apoptotic effects are observed are considerably lower than typically needed to achieve functional inhibition of the sodium pump. These data indicate that cardiac glycosides can selectively induce apoptosis in cancer cells and are a novel effective modality to treat cancer, e.g., malignancies of the immune origin.

The invention will now be illustrated by the following non-limiting Example.

### EXAMPLE 1

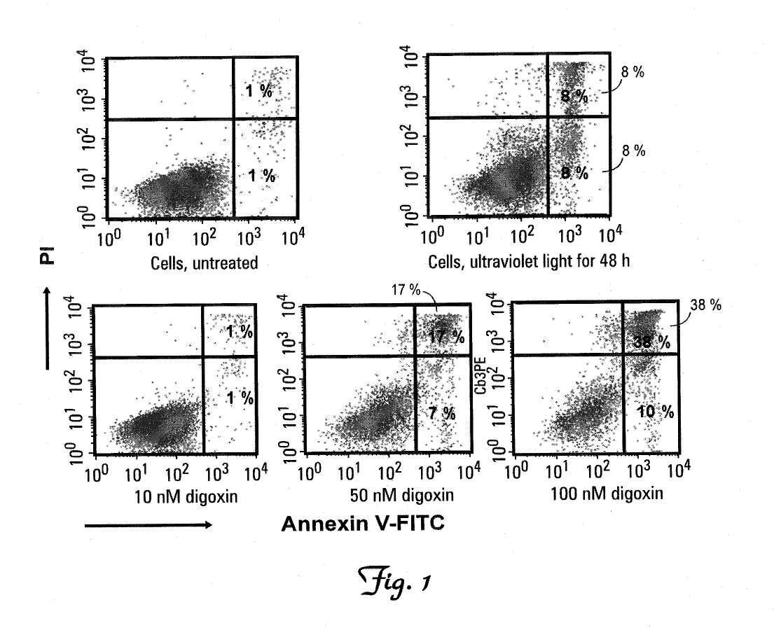

It was determined that the cardiac glycosides (e.g., digoxin and ouabain) selectively induce apoptosis in cancer cells relative to normal non-transformed cells. Through the experiments, some cells were exposed to ultraviolet irradiation as a positive control for induction of apoptosis, whereas cells cultured under normal conditions (95% O₂/5% CO₂ at 37° C.) acted as the negative control (see Methods). A typical flow cytometric analysis is shown in FIG. 1. The percentage of counted cells in the lower and upper right hand quadrants are indicative of cells in early and late apoptosis, respectively. The viable cells are at the lower left quadrant. The figures that follow are summaries of data stemming from analysis of percent of cell count undergoing apoptosis as measured in FIG. 1.

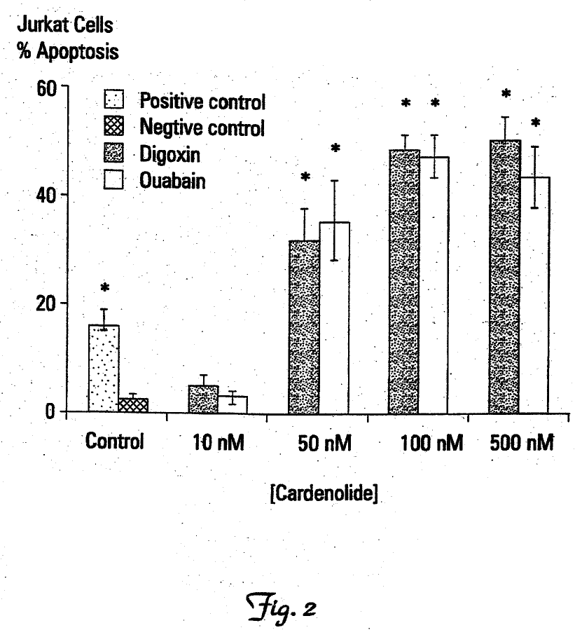

Jurkat cells were exposed to ultraviolet irradiation, digoxin (10 nM-500 nM) or ouabain (10 nM-500 nM) for 48 h. The results shown in FIG. 2 indicate that ultraviolet irradiation significantly (P<0.05) increased apoptosis (16.2%±2.9%, n=4) in Jurkat cells when compared to the untreated control (2.6%±0.9%, n=4). Similarly, exposure to increasing concentration of digoxin (10 nM-500 nM) or ouabain (10 nM-500 nM) significantly (P<0.05) led to increases in apoptosis. Within the concentrations tested, the highest percentage of apoptosis were observed at digoxin (500 nM) (50.2%±4.5%, n=4) and ouabain (100 nM) (47.6±5.6, n=4). Concentrations of those cardiac glycosides above 500 nM did not further significantly increase the number of apoptotic cells.

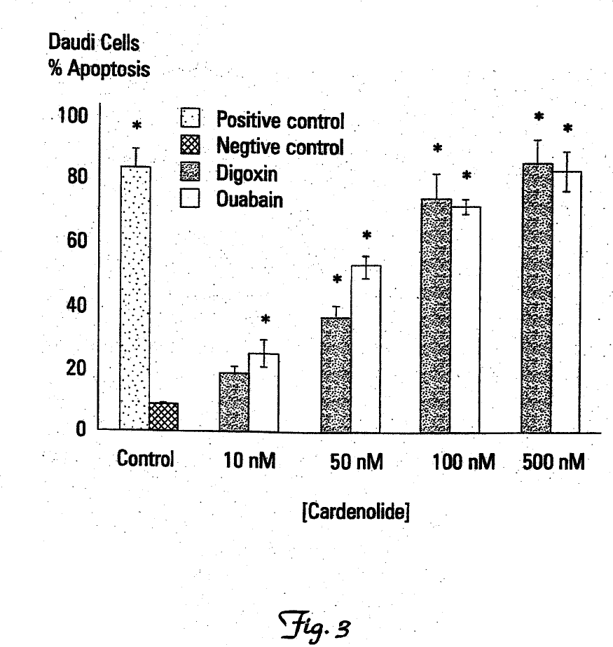

The effects of digoxin and ouabain to induce apoptosis in Daudi cells (5×10⁵ cells/mL) are shown in FIG. 3. Ultraviolet irradiation significantly (P<0.05) increased apoptosis (81.6%±5.6%, n=4) in these cells when compared to the untreated control (7.9%±0.5%, n=4). Similarly, exposure to increasing concentration of digoxin or ouabain significantly (P<0.05) led to increase in apoptosis. For Daudi cells, within the concentration range tested, the highest percentage of apoptosis was observed at digoxin (500 nM-83.2%±7.3%, n=4) and ouabain (500 nM-81.1±6. 1, n=4). Similarly to Jurkat cells, concentrations of digoxin or ouabain above 500 nM did not further significantly increase percentage of apoptotic cells.

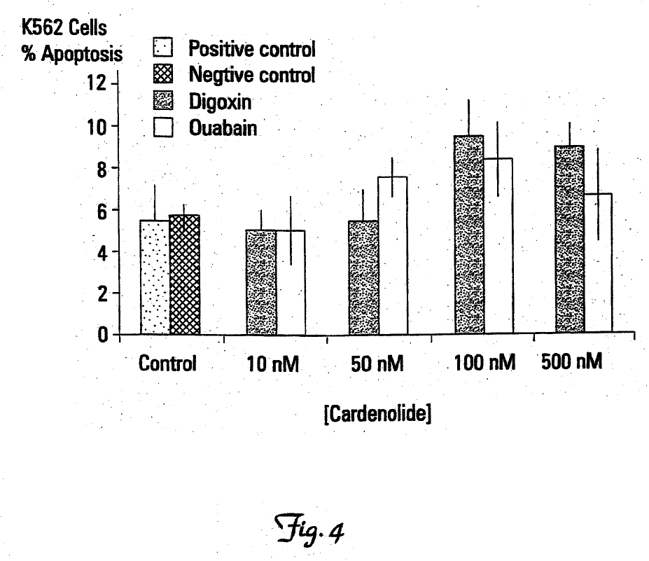

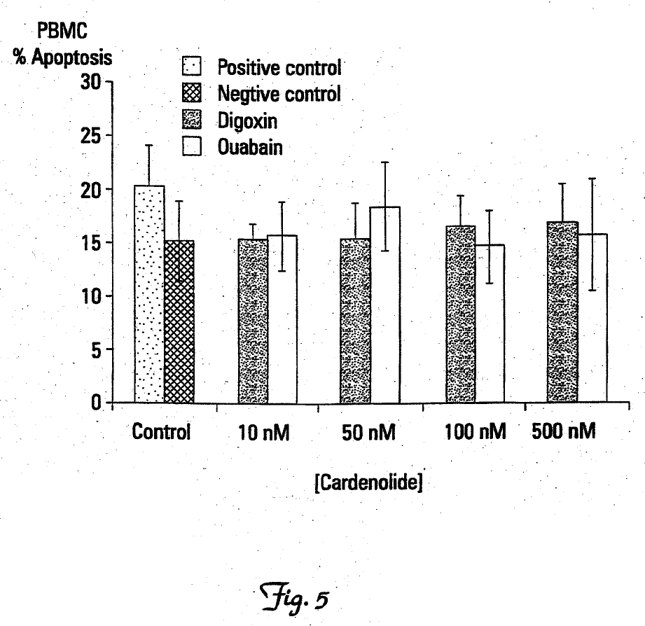

Results for the K 562 cells (5×10⁵ cells/mL) are shown in FIG. 4. The results showed that ultraviolet irradiation did not significantly (P>0.05) increase apoptosis (5.5%±1.7%, n=4) in these cells when compared to the untreated control (5.8%±0.5%, n=4). Similarly, exposure to increasing concentrations of digoxin or ouabain did not significantly (P>0.05) lead to increases in apoptosis. Similar negative results demonstrating no measurable effects on PBMC (1×10⁶ cells/mL) are shown in FIG. 5. These results showed that ultraviolet irradiation did not significantly (P>0.05) increase apoptosis (20.5%±3.8%, n=4) in PBMC when compared to the untreated control (15.3%±4.0%, n=4). Exposure to increasing concentration of digoxin or ouabain did not significantly (P>0.05) lead to increase in apoptosis within the concentration ranges of digoxin and ouabain tested.

In order to compare the concentrations of digoxin or ouabain that affect the induction of apoptosis with those that inhibit the sodium pump, the inhibitory potency of digoxin and ouabain on porcine cerebral cortex (PCC) Na′,K-ATPase catalytic activity was examined. Table 1 summarizes the results on the IC₅₀ of digoxin and ouabain on the inhibition of Na⁺,K⁺-ATPase activity. It is clear they are at least 20-fold higher than that required to induce apoptosis in human lymphoblastic cell lines.

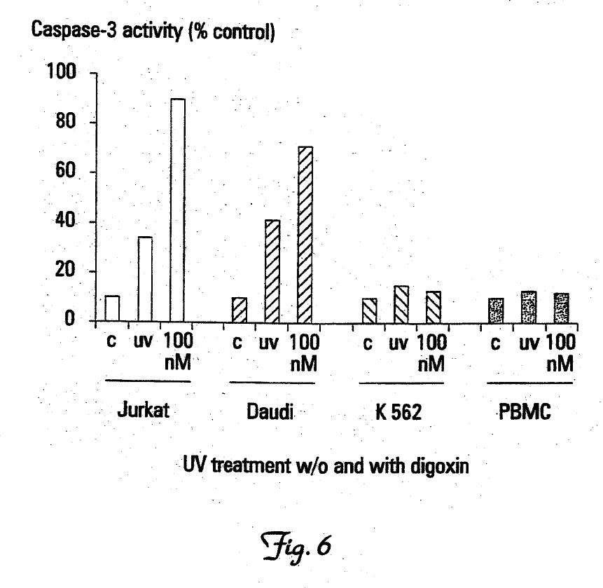

Measurement of DEVD-dependent caspase-3 activity is a measure of induction of apoptosis, irrespective of the apoptotic pathway activated. The effect of digoxin (100 nM) to induce activation of caspase-3 activity in these tumor cell lines and as well as normal PBMC was examined. Tumor cells (1×10⁷ cell/mL) and PBMC (1×10⁷ cells/mL) were exposed to ultraviolet irradiation or digoxin (100 nM) for 12 h. The cells were then analyzed for caspase-3 activation using caspase-3 activation assay kit following the manufacturer's instructions. FIG. 6 show that ultraviolet irradiation and digoxin (100 nM) increased caspase-3 activity in Jurkat cells (2 and 8-fold,) and in Daudi cells (3 and 7-fold) respectively relative to the untreated controls. In contrast, no such increases in caspase-3 activity were observed in K 562 cells or PBMC respectively.

Phytohemagglutinin (PHA) promotes activation-induced apoptosis in T lymphocytes through the FAS/FASL pathway (Martinez-Lorenzo et al., Immunology, 89, 511-517 (1996); Stefan et al., Apoptosis, 5, 153-163 (2000); and Bortner et al., J Biol Chem, 276, 4304-4314 (2001). In order to study the specificity of the effect of cardiac glycosides on induction of apoptosis, Jurkat cells and PBMC were treated with PHA (1 μg/mL) in the presence or absence of digoxin (10 nM-100 nM) for 48 hours. Apoptosis was measured as described herein. In the presence of PHA, digoxin synergistically increased the percentage of apoptosis in Jurkat cells (FIG. 7). There was no such synergistic increase observed in the case of PBMC.

TABLE 1
Comparison of the inhibitory potency of cardiac glycosides on
porcine cerebral cortex Na, K-ATPase catalytic activity and
induction of apoptosis in lymphoblastic cell lines

| | | |
|---|---|---|
| Compounds | Digoxin (n = 5) | Ouabain (n = 5) |

Inhibitory Potency on Porcine cerebral cortex (PCC)
NKA catalytic activity

| | | |
|---|---|---|
| IC₅₀ | 910 nM | 600 nM |
| Range | 820-1010 nM | 500 nM-650 nM |

Induction of apoptosis on acute T-cell lymphoblastic
leukemic cells (Jurkat)

| | | |
|---|---|---|
| IC₅₀ | 24 nM | 26 nM |
| Ranges | 11 nM-56 nM | 19 nM-48 nM |

Induction of apoptosis on B-cell Burkitt's lymphoma
cells (Daudi)

| | | |
|---|---|---|
| IC₅₀ | 48 nM | 40 nM |
| Ranges | 35 nM-65 nM | 36 nM-56 nM |

### PCC, Porcine Cerebral Cortex; NKA, Sodium Potassium ATPase

Comparison of the inhibitory potency of NKA catalytic activity of digoxin and ouabain on PCC and induction of apoptosis on T-cell leukemic cell line (Jurkat) and B-cell leukemic Burkitt's lymphoma cell line (Daudi). The values were calculated as 50% response relative to the maximum NKA activity on the PCC or maximum apoptosis on Jurkat and Daudi cells within the concentration ranges tested. Values represent mean±2 SD, n=5.

### Materials and Methods

All chemicals employed in this study were of reagent grade. Digoxin (Sigma-Aldrich Co. St Louis, Mo.) was dissolved in dimethyl sulfoxide (DMSO; Sigma-Aldrich Co., St Louis, Mo.) and ouabain (Sigma-Aldrich Co. St Louis, Mo.) was dissolved in double distilled water. Both drugs were initially dissolved to a concentration of 10 mmol/L and stored at −20° C. These drugs were further dissolved in cell culture medium for in vitro studies. All reagents employed for inhibition of Na⁺,K⁺-ATPase catalytic activity (ATP, ammonium molybdate, Tween-80 and bovine serum albumin) were purchased from Sigma-Aldrich Co. (St. Louis, Mo.). Phytohemagglutinin (PHA) (Sigma-Aldrich Co, St Louis, Mo.) was dissolved in cell culture medium to a concentration of 1 mg/L.

Cell lines used in this study included: Jurkat E6-1, an acute human T-lymphoblastic leukemia cell line generated from a 14-year-old male; Daudi, a human B-lymphoblastoid line derived from Burkitt's lymphoma patient and K 562 derived from a human Caucasian chronic myelogenous leukemia cell line. All cell lines were maintained in RPMI 1640 medium (Gibco Laboratories, Grand Island, N.Y.) supplemented with 10% fetal calf serum (FCS), 10 mM HEPES, 2 mM glutamine, 50 IU/mL penicillin, 50 μg/mL streptomycin, 0.1 mM non-essential amino acids and 1 mM sodium pyruvate and 0.5 μg/mL amphotericin B. Cells were cultured in a 5% CO₂ atmosphere in a thermostatically maintained incubator (37° C.) in standard cell culture flasks. Cell cultures were split every 2-3 days and passage number noted.

Peripheral blood mononuclear cells (PBMC) were isolated from heparinized blood obtained from healthy consenting volunteers by density gradient centrifugation using Histopaque R-1077 (Sigma-Aldrich Co., St Louis, Mo.), as described previously (Ihenetu et al., Eur J Pharmacol, 464, 207-215 (2003)). In brief, whole human heparinized blood was diluted (1:2) in sterile phosphate buffered saline (PBS), layered over Histopaque and PBMC were isolated following gradient centrifugation (800×g for 30 min) in an Accuspin tube (Sigma-Aldrich Co., St. Louis, Mo.). Cells, recovered from the interface between plasma and Histopaque solution, were washed twice in Ca²⁺ and Mg²⁺ free PBS (250×g for 10 min). PBMC were resuspended in RPMI 1640 supplemented with L-glutamine (2 mM), penincillin (50 U/mL) and streptomycin (50 μg/mL) and 10% heat inactivated FCS. Aliquots of the cells were removed for cell counting in a Neubauer counting chamber and assayed for viability by trypan-blue dye exclusion method. Slides of the cell suspension were made and stained by Romanowsky stain (May Grunwald-Giemsa) and a differential cell count was obtained.

Inhibition of sodium pump catalytic activity by cardiac glycosides was measured by the release of phosphate upon hydrolysis of ATP (Qazzaz et al., Endocrinology, 141, 3200-3209 (2000)).

Cell viability was measured using MTT assay (Ihenetu et al., Eur J Pharmacol, 464, 207-215 (2003)). Briefly, cells were cultured at a density of 1×10⁵ cells/well in a 96 well plate with different concentrations of digoxin or ouabain. At the end of the incubation period (12 h), media were removed and 10 μl/mL of MTT reagent (5 mg/mL) was added to all wells and incubated at 37° C. for 2 h. Acidic-isopropanol (100 μl/mL) was added to each well and thoroughly mixed to dissolve the dark crystals. Absorbance was measured at 570 nm wavelength and results were expressed as % of control values.

Five volumes of trypan blue dye (0.4% in PBS) were mixed with 1 volume of cells in suspension and incubated at room temperature for 5 min. The cell suspension was then counted in an improved Neubauer counting chamber. All counts were performed in duplicate. Cell viability was expressed as % of cells that excluded the dye from the total number of cells counted.

Tumor cell lines (5×10⁵ cells/well) and PBMC (1×10⁶ cells/well) were cultured in 24-well plates in the presence or absence of various concentrations of digoxin or ouabain for 48 h. The cells were harvested, washed twice in PBS, and analyzed for induction of apoptosis by annexin V-FITC/propidium iodide (PI) method (BD Bioscience, Lincoln Park, N.J.) according to the manufacturer's instructions. Cells were washed once with 133 binding buffer and stained with annexin V-FITC (5 μL) and PI (10 μL) for 15 minutes in the dark. Apoptosis was determined by flow cytometric analyses on a FACScan (BD Biosciences, Lincoln Park, N.J.). Ten thousand cells were analyzed per sample. In experiments where the effect of PHA was studied, cells were seeded accordingly in a 24-well plate and stimulated with PHA (1 μg/mL) for a minimum of 2 hours before treatment with the indicated concentrations of ouabain or digoxin. As a positive control for apoptosis, cells were exposed to ultra violet irradiation for 48 hours and apoptosis was analyzed according to the method described above.

Caspase-3 activity was measured using a caspase-3 assay kit (Sigma-Aldrich Co. St Louis, Mo.). Briefly, tumor cell lines (1×10⁷ cells/mL), and PBMC (1×10⁷ cells/mL) were cultured in the presence or absence of the indicated concentration of digoxin. Cells were harvested by centrifugation and washed once with PBS. Cells were lysed with lysis buffer (30 mM Tris-HCl, pH 7.5, 150 mM NaCl, 1% Triton X-100, and 10% glycerol) and were centrifuged to remove cell debris, Caspase 3 activity was assayed in the cell lysate according to the manufacturer's instructions. The colorimetric assay is based on spectrophotometric detection of chromophore pNA at 405 nm after cleavage from labelled substrate DEVD-pNA. The level of caspase 3 activity is interpolated from a calibration curve.

Human peripheral blood mononuclear cell preparations from healthy volunteers included approximately 95% lymphocytes and 5% monocytes as measured by differential leukocyte counts. Under our experimental conditions, the viability of human peripheral blood mononuclear cells isolated from heparinized blood obtained from healthy volunteers exceeded 95% on all experiments, when determined by trypan blue dye exclusion method and MTT assay respectively.

Statistical evaluation was performed using Student's t-test. Differences were considered significant at P≦0.05.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for treating cancer in a subject, comprising administering to the subject an effective amount of a cardiac glycoside so as to treat the cancer.

2. A method for inducing cellular apoptosis of a cancerous cell, comprising contacting the cancerous cell with an effective apoptosis-inducing amount of a cardiac glycoside.

3. A method for increasing the anticancer effects of a cancer therapy on a cancerous cell, comprising contacting the cancerous cell with an effective amount of a cardiac glycoside prior to administering the cancer therapy.

4. The method of claim 1, wherein the cardiac glycoside is a cardenolide.

5. The method of claim 4, wherein the cardenolide is a digoxigenin, digoxin, dihydrodigoxin, digitoxigenin, digitoxin, neriifolin, strophanthidin, convallatoxin, acetylstrophanthidin, ouabagenin, or ouabain.

6. The method of claim 4, wherein the cardenolide is a mammalian cardenolide.

7. The method of claim 6, wherein the mammalian cardiac glycoside is a digoxin-like factor (DLF), digoxin-like immunoreactive factor (DLIF), ouabain-like factor (OLF), dihydroouabain-like factor (Dh-OLF), or dihydrodigoxin-like factor (Dh-DLIF).

8. The method of claim 1, wherein the cardiac glycoside is a bufadienolide.

9. The method of claim 8, wherein the bufadienolide is a bufalin, proscillardin, marinobufagenin, cinobufagen, or cinobufatolin.

10. The method of claim 2, wherein the contacting step occurs in vivo.

11. The method of claim 2, wherein the contacting step occurs in vitro.

12. The method of claim 1, further comprising administering an additional cancer therapy to the subject.

13. The method of claim 2, further comprising administering an additional cancer therapy to the cell.

14. The method of claim 12, wherein the additional cancer therapy is chemotherapeutic or radiation.

15. The method of claim 1, wherein the effective amount of the cardiac glycoside does not significantly inhibit the activity of a sodium pump.

16. The method of claim 1, wherein the cancer is breast cancer, prostate cancer, lung cancer, colon cancer, hepatic cancer, skin cancer, leukemia, or lymphoma.

17. A pharmaceutical composition comprising an effective anticancer amount of a cardiac glycoside and a pharmaceutically acceptable carrier.

18. The pharmaceutical composition of claim 17, wherein the cardiac glycoside is a cardenolide.

19. The pharmaceutical composition of claim 18, wherein the cardiac glycoside is a mammalian cardenolide.

20. The pharmaceutical composition of claim 17, wherein the cardiac glycoside is a bufadienolide.
